(19) United States
(12) Patent Application Publication
(10) Pub. No.: US 2003/0036759 A1
(43) Pub. Date: Feb. 20, 2003

(54) Modular spinal plate system

(76) Inventor: Emilio Musso, Boca Raton, FL (US)

Correspondence Address: MCHALE & SLAVIN, 4440 PGA BLVD, SUITE 402, PALM BEACH GARDENS, FL, 33410

(21) Appl. No.: 09/929,906

(22) Filed: Aug. 14, 2001

(51) Int. Cl. A61B 17/80

(52) U.S. Cl. 606/69

(57) ABSTRACT

A modular spinal plate system for anterior fixation of the cervical spine has several anchoring washers attached to vertebrae by pedicle screws. A thin flat spinal plate sized to span at least two vertebrae and including elongated apertures is placed over the anchoring washers. The plate is fastened to the washers by threaded connections through the elongated apertures to maintain the vertebrae in a fixed spatial orientation.

### FIELD OF THE INVENTION

This invention relates to the field of orthopedic surgery and spinal fixation. The apparatus of this invention is attached to two or more vertebrae and maintains them in fixed spatial orientation to each other.

### BACKGROUND OF THE INVENTION

Spinal fixation is a conventional surgical procedure for fixing the position of several vertebrae in relation to each other to prevent movement between the vertebrae. The procedure may be in conjunction with a graft between the vertebrae which ultimately fuses the vertebrae together with body tissue. During the period of time required for the graft to become functional, the vertebrae are stabilized in a fixed orientation by rods or plates, usually made of metal, connected to the spine by pedicle screws. The fixation may be performed in the cervical, thoracic or lumbar regions of the spine. The plates or rods may be placed on the dorsal or anterior surface of the vertebrae.

Spinal plates are normally used for anterior placement in the cervical region because of the low profile necessitated by the proximity of vital soft tissue organs to the spine. The spinal plates now used come in various sizes, of course, but the main criteria for selection is the number of vertebrae to be stabilized. The plates are basically rectangular with a series of paired apertures along the opposite long sides. These paired apertures allow bone screws to be placed in each vertebra to fasten several vertebrae rigidly to the plate. Once the decision is made as to the number of vertebrae involved, the surgical procedure is dictated by the shape of the plate.

These spinal plates present several problems related to the fact that the human or animal skeleton is non-uniform and the vertebrae do not necessarily line up, vertically or laterally, with the paired apertures. This results in compromising the position of the plate to obtain the most suitable location for insertion of the bone screws in each vertebra. In the end, the position of the plate and the location of the bone screws in the vertebrae is not optimal.

Also, during the operation, once the plate is placed on the vertebrae the visualization of the vertebrae is lost. This can lead to a misalignment of the vertebrae and/or misplacement of a screw(s) in vertebrae.

The above problems are further exacerbated in the anterior placement of the cervical spinal plate because of the limited surgical field. The surgical approach for anterior placement does not present a head-on view of the spine. The incision is in the neck, between the larynx and the carotid artery, which limits retraction. The elongated plate may be inserted through a shorter incision and maneuvered to align it with the spine. Once the first hole is drilled in a vertebra much of the flexibility with regard to the remaining vertebrae is lost.

### DESCRIPTION OF THE PRIOR ART

U.S. Pat. No. 6,176,861 to Bernstein et al discloses a modular spinal fixation system for dorsal placement in the cervical, thoracic and lumbar regions of the spine. The system includes a plurality of pedicle screws which have an offset arm carrying a screw connector. Each pedicle screw is placed in a vertebra and the offset arm is manipulated to compensate for lateral irregularities in the spine. The screw connectors are threaded in the offset arm to compensate for vertical irregularities. An elongated oval plate having a central opening is placed over the plurality of screw connectors. Head pieces are screwed onto the connectors fastening the plate and the vertebrae in a fixed position.

U.S. Pat. No. 5,129,899 to Small teaches another modular system with several pedicle screws placed in separate vertebra. An elongated plate with a continuous central slot is placed over the several pedicle screws. The outer periphery of the elongated plate has serrations which cooperate with keyed washers placed over the protruding ends of the pedicle screws and the periphery of the plate. These washers are locked in place by threaded caps screwed onto the ends of the pedicle screws.

The Lowery et al patent, U.S. Pat. No. 5,364,399, teaches the use of a spinal plate for anterior fixation in the cervical region. The patent enumerates the problems of the anterior placement of a fixation device.

What is needed is a modular, low-profile spinal plate system that permits visualization of the vertebrae and optimizes the placement of the pedicle screws in each vertebra while reducing trauma to the surrounding surgical site by reducing the size of the components.

### SUMMARY OF THE INVENTION

Accordingly, it is an objective of the instant invention to provide a modular spinal plate system for fixation of several vertebrae by attachment of separate anchoring washers to the anterior surface of the vertebrae.

It is a further objective of the instant invention to provide a low-profile spinal plate which cooperates with several anchoring washers to maintain the spatial orientation between the separate anchoring washers.

It is yet another objective of the instant invention to teach the use of locking screw assemblies to fasten the anchoring washers and spinal plate together.

It is a still further objective of the invention teach the use of the spinal plate as a back plate to prevent loosening of the anchoring washer attachment to the vertebrae.

Other objects and advantages of this invention will become apparent from the following description taken in conjunction with the accompanying drawings wherein are set forth, by way of illustration and example, certain embodiments of this invention.

### DESCRIPTION OF THE FIGURES

FIG. 1 shows an exploded perspective of the modular spinal plate system of this invention;

FIG. 2 shows a perspective of an anchoring washer;

FIG. 3 shows a longitudinal cross section through several anchoring washers of the spinal plate system;

FIG. 4 shows a lateral cross section through one anchoring washer; and

FIG. 5 shows an exploded perspective of a modification of the of the spinal plate and anchoring washer.

### DESCRIPTION OF THE INVENTION

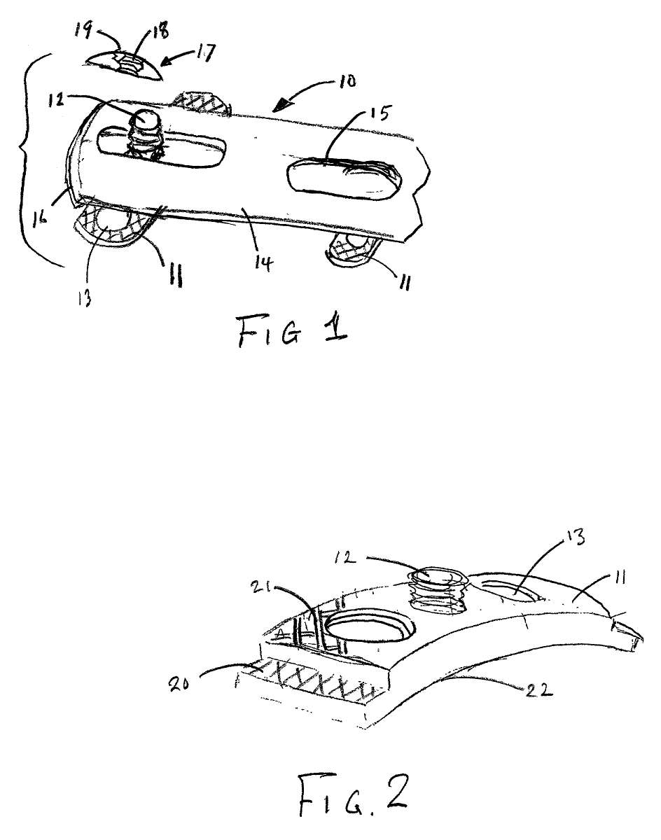

In FIG. 1, the modular plate system 10, for use in the cervical region of the spine has anchoring washers 11 which are to be attached to the vertebrae 30 by pedicle screws 40, shown in FIG. 3. The pedicle screws 40 are inserted through the apertures 13 that may or may not be tapped and threaded into the spine. The apertures are countersunk to accept the heads of the pedicle screws, as shown in FIG. 4. Each anchoring washer 11 has a threaded post 12. The modular construction of the anchoring washers permits the optimal placement of the pedicle screws in each vertebra.

After a series of anchoring washers are attached to the anterior surfaces of the vertebrae, an elongated spinal plate 14 is placed over the washers, as shown in FIG. 3. The plate 14 has a series of large openings 15 along it's length. A portion 16 of a depending flange forms a stop for limiting longitudinal movement of the components. The depending flange may be omitted, if desired, for more flexibility in matching the components.

The posts 12 of the anchoring washers are inserted through an elongated opening 15 and a locking screw assembly 17 is threaded onto each post by cooperating internal threads in aperture 18, as shown in FIG. 3. The elongated openings 15 are larger than the diameter of the posts 12 to compensate for the non-uniformity in the vertebrae. The width of the opening 15 allows for lateral non-alignment of the posts 12 and the length of the openings allows for differences in vertical or longitudinal spaces between vertebrae. The diameter of the locking screw assembly 17 is greater than the width of the elongated opening 15. As the locking screw assembly is threaded onto the post 12 compressive force is applied to the spinal plate 14 and anchoring washer 11 to fasten them together. The locking screw assembly 17 is locked in place by set screw 19 to prevent loosening of the assembly. The resulting rigid connection of the anchoring washers and the spinal plate holds the attached vertebrae in a fixed spatial orientation.

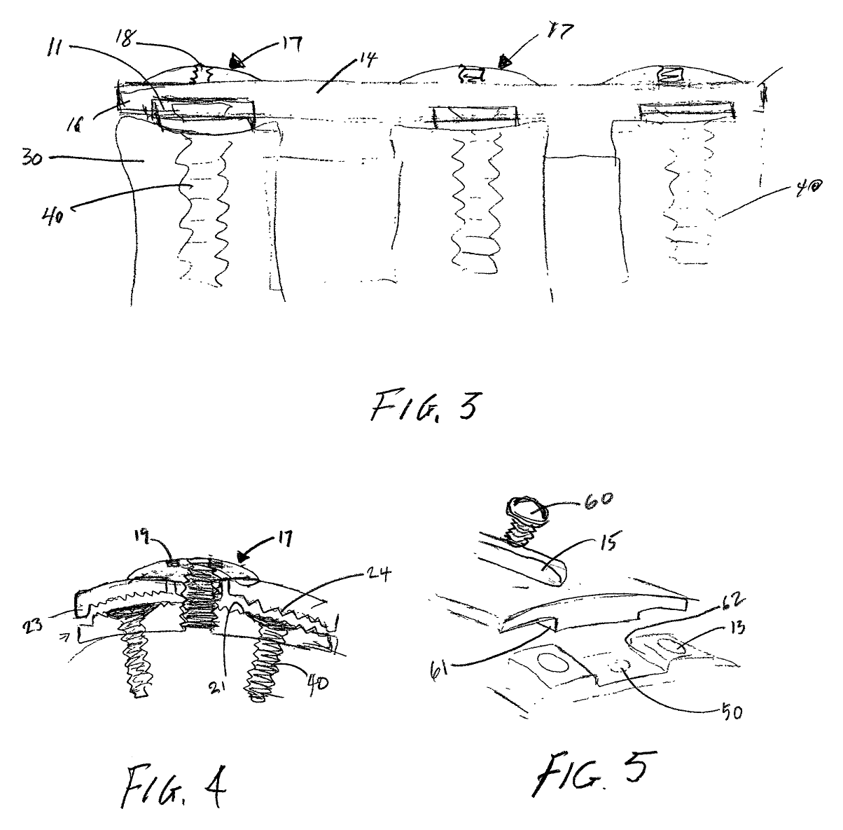

The low-profile required by the anterior approach is shown in FIG. 2. To increase the amount of surface area in interlocking relationship, the anchoring washer 11 may be formed with thinner flanges 20 along the opposite ends. These flanges may cooperate with a longitudinal depending flange 23 on the spinal plate, shown in FIG. 4, to form a more secure connection between the anchoring washers and the spinal plate. Of course the depending flange 23 may be omitted, particularly in the modifications in which the spinal plate does not extend completely across the anchoring washers. The entire thickness of the system is composed of the thickness of the anchoring washer, the flat plate and the exposed threads of the post. Because of the rotational movement of the neck and cervical area of the spine, the anterior surface of the system components are smooth and without protrusions.

There is a tendency for the pedicle screws to work loose in response to the varying forces placed on the vertebrae during the normal movement of the body. The spinal plate 14 has a width such that the longitudinal edges overlie at least a portion of the heads of the pedicle screws to act as a back plate or stop, as shown in FIG. 1 and FIG. 5.

FIG. 5 illustrates another embodiment of the spinal plate system in which the anchoring washers and the plate are fastened together by a bolt 60 threaded through a tapped aperture 50, rather than the post of FIG. 1. Also shown is another embodiment of the cooperating flanges. A central thickened elongated portion 61 on the plate fits into a central groove 62 in the washers to increase stability. The cooperating patterns of lands and grooves 21 and 24 may also be utilized in this configuration.

To further increase the interlocking relationship of the anchoring washers and the plate, the upper surface of the anchoring washers have a pattern of lands and grooves 21 which cooperate with a similar pattern of lands and grooves 24 on the lower surface of the spinal plate. When the plate and the washers are superimposed, these patterns prevent lateral or longitudinal relative movement.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

What is claimed is:

1. A modular orthopedic plate system for anterior application to the cervical spine for fixation of vertebrae, said system comprising (a) a plurality of anchoring washers, each of said plurality of anchoring washers having a minor thickness connecting opposite distal and proximal major surfaces, said major surfaces being of a size and shape approximating the anterior surface of a vertebra, at least two apertures through said minor thickness of each of said plurality of anchoring washers, said distal major surface of said plurality of said anchoring washers for contacting a vertebra, said proximal major surface of each of said plurality of anchoring washers including connectors near the center of said proximal surface between said apertures, and (b) an elongated vertebral plate of a length to span at least the distance between the centers of two adjacent vertebrae, said plate having at least two apertures spaced along said length, said apertures sized to accommodate and receive said connectors and adjust for longitudinal and lateral displacement of said vertebrae, whereby each of said plurality of anchoring washers are adapted to be attached to a vertebra, said vertebral plate is adapted to contact said proximal major surface of said anchoring washers with said anchoring washer connectors disposed in said vertebral plate apertures and applying compressive force to fix a plurality of vertebrae in place relative to each other.

2. A modular orthopedic plate system of claim 1 wherein said vertebral plate has a distal surface for contacting said proximal surface of said plurality of anchoring washers, said distal surface of said plate and said proximal surface of each of said anchoring washers have a pattern of lands and grooves adapted to interlock upon contact of said surfaces preventing lateral or longitudinal relative movement.

3. A modular orthopedic plate system of claim 2 wherein said elongated vertebral plate has a width at least equal to the distance between said apertures in each of said anchoring washers whereby said vertebral plate overlies a portion of each of said apertures.

4. A modular orthopedic plate system of claim 3 wherein said apertures in each of said anchoring washers are countersunk on said proximal major surface to accommodate the head of a bone screw.

5. A modular orthopedic plate system of claim 4 wherein said elongated vertebral plate has a longitudinal edge, said longitudinal edge having a depending flange extending toward said proximal surface of each of said anchoring washers, each of said anchoring washers have a thinner shelf on said proximal major surface, said depending flange adapted to contact said thinner shelf.

6. A modular orthopedic plate system of claim 5 wherein said depending flange extends from the longitudinal center portion of said vertebral plate and said thinner shelf of each of said anchoring washers is between said apertures.

7. A modular orthopedic plate system of claim 1 wherein said connectors are threaded posts extending from the proximal major surfaces of said anchoring washers and a locking screw assembly is threadably secured to said posts to secure said post to said vertebral plate.

8. A modular orthopedic plate system of claim 7 wherein said elongated vertebral plate has a width at least equal to the distance between said apertures in each of said anchoring washers whereby said vertebral plate overlays a portion of each of said apertures.

9. A modular orthopedic plate system of claim 7 wherein said apertures in each of said anchoring washers are countersunk on said proximal major surface to accommodate the head of a bone screw.

10. A modular orthopedic plate system for anterior low profile fixation of the cervical spine, said system comprising a plurality of planar anchoring washers adapted to be optimally positioned and serially attached to several cervical vertebrae by bone screws, said anchoring washers each having at least three apertures, one of said apertures located near the center of each anchoring washer being internally threaded, an elongated planar vertebral plate a length to span at least two vertebrae, said plate having a plurality of oblong apertures spaced along said length, said oblong apertures being of a dimension to accommodate lateral and longitudinal displacement of said plurality of anchoring washers, said plate having a width at least equal to the distance between said at least three apertures, and a plurality of lock screws each having a head larger than said dimension of said oblong apertures, said lock screws each having threads complementary to the internal threads in said internally threaded apertures in said anchoring washers, whereby said vertebral plate is adapted to be placed in contact with said plurality of optimally positioned anchoring washers with said threaded apertures in said anchoring washers aligned with the oblong apertures of said vertebral plate and said locking screws are adapted to be inserted through said oblong apertures and threaded into said threaded apertures securing said anchoring washers and said vertebral plate together by compression.

11. A modular orthopedic plate system of claim 10 wherein said anchoring washers each have a distal surface for contact with a vertebra and a proximal surface with a pattern of lands and grooves, said vertebral plate has an elongated distal surface for contact with said proximal surface of said anchoring washers, said distal surface of said vertebral plate having a complementary pattern of lands and grooves whereby said anchoring washers and said vertebral plate are interlocked by said lands and grooves to prevent lateral or longitudinal relative movement.
